(19) United States
(12) Patent Application Publication
(10) Pub. No.: US 2004/0002644 A1
(43) Pub. Date: Jan. 1, 2004

(54) Method and apparatus for digitizing electromagnetic radiation measurements by shutter speed control

(75) Inventors: Thorstein Seim, Slependen (NO); Roland Eisentraeger, Saetre (NO)

Correspondence Address: LAW OFFICE OF BARRY R LIPSITZ, 755 MAIN STREET, MONROE, CT, 06468, US

(73) Assignee: Axis-Shield PoC AS, Oslo (NO)

(21) Appl. No.: 10/189,287

(22) Filed: Jul. 1, 2002

(51) Int. Cl. A61B 5/05

(52) U.S. Cl. 600/407

(57) ABSTRACT

This invention relates to a method, device and apparatus for digitizing electromagnetic radiation measurements by control of camera shutter speed. The invention uses an Electromagnetic Radiation Sensitive Device (ERSD), such as for example a camera system containing a CMOS- or a CCD-image chip, to perform precise measurements by high-resolution digital control of the shutter speed. A constant output value is obtained from the ERSD such that any non-linearity and range limitation of the ERSD output is circumvented. The measurement methods and system are applied to chemical tests and analytes, which are used for diagnostic purposes. The method can be used to measure reflectance, transmittance, fluorescence and turbidity.

### BACKGROUND OF THE INVENTION

1. Field of the Invention

This invention relates to the field of measurement technology. More specifically, the invention relates to a method and apparatus for digitizing electromagnetic radiation measurements by a high-resolution control of camera shutter speed.

2. Description of the Related Art

In electromagnetic radiation measuring instruments with built-in electromagnetic radiation source the electromagnetic radiation level is normally kept at a constant level and is turned on and off according to the process performed by the instrument. An electromagnetic radiation sensitive device in the instrument is usually adjusted until it is able to properly detect the amount of electromagnetic radiation from a test and/or reference object. Other imaging systems, not fitted with an electromagnetic radiation source, are adjusted to the ambient electromagnetic radiation level. An example is the photographic (film) camera. In order to expose the film correctly the shutter speed and lens aperture are adjusted, usually after measuring the electromagnetic radiation (usually visible light) from the test object with an electromagnetic radiation meter. Digital cameras are also constructed to be able to measure and use the ambient light. For these cameras the light meter is usually the electromagnetic radiation sensitive image chip itself. Digital cameras normally contain an electronic shutter, which is used to adjust the amount of electromagnetic radiation recorded.

Problem to be Solved by the Invention

Inexpensive digital cameras, like those used as web-cameras, are normally not used in precision electromagnetic radiation measurement instruments. They tend to have limited output resolution range. In addition the signal output tends to be a non-linear function of the received electromagnetic radiation intensity.

U.S. patent application Ser. No. 09/952,382, by the same inventor as the present application, solves the problem with non-linear electromagnetic radiation sources by a method and system that controls the intensity of the electromagnetic radiation source to achieve a constant output from the electromagnetic radiation sensitive device, and this application is incorporated herein by reference.

However, the measuring range and the measurement accuracy of such cameras mentioned above can be improved by a high-resolution control of the shutter speed. Nearly all cameras, for example those used for digital video, works in a similar way. The pixels in each line of the camera-chip are charged to a given voltage at a time T₀. After charging is finished the electromagnetic radiation quanta, absorbed by the pixels, will lead to an electric current that de-charge the pixels. The more electromagnetic radiation the faster the discharge will be. After a time T₁ (the shutter speed) the charge (or voltage) of the pixels in a line is transferred to an output circuit and digitized. Normally the T₁ timing is set as a given number of line periods.

The line period time is usually 64 μs in a standard PAL TV image for example, but in most cameras the shutter speed can electronically be adjusted down to a few μs. The shortest time is obtained by charging the line-pixels one line before readout. The next exposure step is obtained by adding the time of one line to the delay, thereby increasing the exposure time by 100%. However, if the time between lines can electronically be adjusted with a small amount, say Δt=50 ns, a precise and flexible intra-line exposure time can be introduced in the camera thereby permitting use of the camera for example for exposure until a fixed target value is obtained from the pixels with a resolution determined by the Δt time.

Means for Solving the Problem

The invention solves the aforementioned problem by using an Electromagnetic Radiation Sensitive Device (ERSD), such as for example a camera system containing a CMOS- or a CCD-image chip, to perform precise measurements by digitally controlling the shutter speed of the CCD camera chip (CMOS: Complementary Metal-Oxide Semiconductor; CCD Charge Coupled Device). A constant output value is obtained from the ERSD such that any non-linearity and range limitation of the ERSD output is circumvented. The measurement methods and system are applied to chemical tests and analytes, which are used for diagnostic purposes. The method can be used to measure reflectance, transmittance, fluorescence and turbidity in accordance with U.S. patent application Ser. No.: 09/952,382.

These and other objects and features of the invention are provided by a method for control of the shutter speed, a system using said method, and a search-method to obtain the measurement result quickly is presented.

### BRIEF SUMMARY OF THE INVENTION

The present invention comprises a method for digitizing electromagnetic radiation to recorded from an illuminated test object by controlling the shutter speed. The electromagnetic radiation from the test object is recorded. The shutter speed of the camera chip is varied until a requested Target output from the ERSD is obtained. If the test object is changed, the amount of electromagnetic radiation from it will normally also change. The shutter speed is then changed until the ERSD output again is equal, or nearly equal to the Target value. The necessary adjustment of the value for the shutter speed controller is used to compute the amount of electromagnetic radiation from each test object. Thus the effect of limited range and non-linearity of an ERSD can be circumvented.

The method of digitizing electromagnetic radiation levels by successive approximation to measure an electromagnetic radiation value, comprises:

Identifying an output target value of an electromagnetic radiation sensitive device receiving electromagnetic radiation signals modified by a test object;

Defining an initial step value of an analog to digital converter (ADC) connected to a part of the electromagnetic radiation sensitive device;

Setting the initial step value to be the value of the Δt shutter time controlling a shutter speeds wherein the shutter settings has an N bit resolution;

Repeating one or more shutter tine adjustments and corresponding Δt time shutter speeds based on a relationship between the shutter value and the ADC value of the output target value for up to N−1 iterations while using corresponding Δt time adjustments until the ADC value is equal to the output target value when the adjustments are completed; and

Identifying the final shutter value (final corresponding Δt time adjustment value) as a measure of the value of the electromagnetic radiation signals.

For example, with a shutter speed adjustment step value of Δt=50 ns, a pixel line exposure time of 64 μs per line (shutter speed), the pixel line will be divided into 1280 exposures of 50 ns each, which will provide an increase of the total resolution of the image from the electromagnetic radiation sensitive device by aproximately 10 bits.

The present invention furthermore disclose a method of digitizing electromagnetic radiation measurements by controlling a shutter speed, to obtain a constant or near constant signal from the electromagnetic radiation sensitive device, the method comprising:

Illuminating an illumination region by a plurality of electromagnetic radiation signals;

Modifying the plurality of electromagnetic radiation signals;

Recording the plurality of modified electromagnetic radiation signals;

Transmitting an output signal corresponding to the plurality of modified electromagnetic radiation signals; and

Calculating the Δt adjustments of shutter speed based on the output signal, such that the output signal is constant.

The present invention also comprises a system for digitizing electromagnetic radiation measurements by controlling a shutter speed, to obtain a constant or near constant signal from said electromagnetic radiation sensitive device. The system comprises:

An electromagnetic radiation source illuminating an illumination region, having a test object, by a plurality of electromagnetic radiation signals;

An electromagnetic radiation sensitive device configured to record the plurality of electromagnetic radiation signals generally modified by the test object in the illumination region and transmit an output signal corresponding to the modified plurality of electromagnetic radiation signals;

A data processor system configured to receive the output signal and generate a controlling signal; and

A Δt shutter speed controller, receivable connected to the data processor system via the controlling signal, the Δt shutter speed controller controlling the operation of the shutter, whereby the received electromagnetic radiation signals are adjustably controllable such that said output signal is constant.

In an alternative embodiment, the system comprises:

A data processor system configured to generate a controlling signal;

A Δt shutter speed controller responsive to the controlling signal;

An electronic shutter responsive to the Δt shutter speed controller;

An illumination region, including a test object, illuminated by the electromagnetic radiation source; and

An electromagnetic radiation sensitive device, configured to image the electromagnetic radiation modified by the test object, and communicate an output signal representative of the modified electromagnetic radiation to the data processor system, whereby the modified electromagnetic radiation signal is adjustably controllable such that the output signal is constant.

The method and apparatus of the present invention can be used to determine the characteristics of an unknown electromagnetic radiation source. By using a known reference source and removing the Target object from the system, the system be calibrated with this known source permitting an unknown source to be identified after replacing the known source with the unknown source in the system.

The present invention also comprises a method for controlling a shutter speed in a camera-chip, the method comprising;

A counter is reset and the clock of the camera-chip is halted when a pixel line has finished being exposed (shutter speed time).

The counter counts the clock pulses and when the counter reaches a predefined value, the halting of the camera clock is removed and the camera-chip is again clocked until the next pixel line has been exposed.

The present invention also includes a device performing the above-mentioned method for controlling the shutter speed. The functional blocks and their interactions of said device are depicted in FIG. 3.

Electromagnetic radiation from the test object is received by an ERSD, for example a digital camera, and the Analog-to-Digital output Converter (ADC) of the camera is connected to the microprocessor system. The computer system can then adjust the Δt shutter time until a given Target value output from the ERSD is obtained by comparing the received digitized electromagnetic radiation values, and by comparing with the target value, iterate the Δt dime shutter speed until the target value is reached. The procedure can be performed by using a single picture element pixel) in the camera image of the test object or a group of pixels. Reflected, transmitted, re-transmitted (as for fluorescence) and/or diffused electromagnetic radiation from the test object can be measured by this method.

Shutter speed adjustments corresponding to Δt adjustments to obtain the Target value are done by a successive approximation search-method. The number of shutter speed adjustment steps in this method will then define the resolution (number of bits) in the answer. The number of bits is also equal to the number of shutter speed settings and subsequent readings of ADC values. However, the search can be executed faster: By initially calibrating the system set-up (with a Reference test object), a faster search can be performed by doing a fast search in the calibration table, combined with necessary numbers of image capture.

### BRIEF DESCRIPTION OF THE DRAWINGS

FIG. 1 illustrates the system set-up according to an embodiment of the invention, using the method in accordance with the invention. The system uses a microprocessor system to control the shutter speed of the camera. The electromagnetic radiation source illuminates a test object. An Electromagnetic Radiation Sensitive Device receives electromagnetic radiation from the test object. The output from the device is received by the processor system.

FIG. 2 illustrates an example of how the analog output of an Electromagnetic Radiation Sensitive Device can be digitized.

FIG. 3 shows the control device for camera pixel exposure control.

FIGS. 4-6 are flowcharts illustrating the successive approximation method (SAM) applied for digitization of electromagnetic radiation levels using Δt time shutter speed iterations, FIG. 4 illustrating a single pixel SAM, FIG. 5 illustrating a meta-pixel SAM, FIG. 6 illustrating a fast meta-pixel SAM.

### DETAILED DESCRIPTION OF THE INVENTION

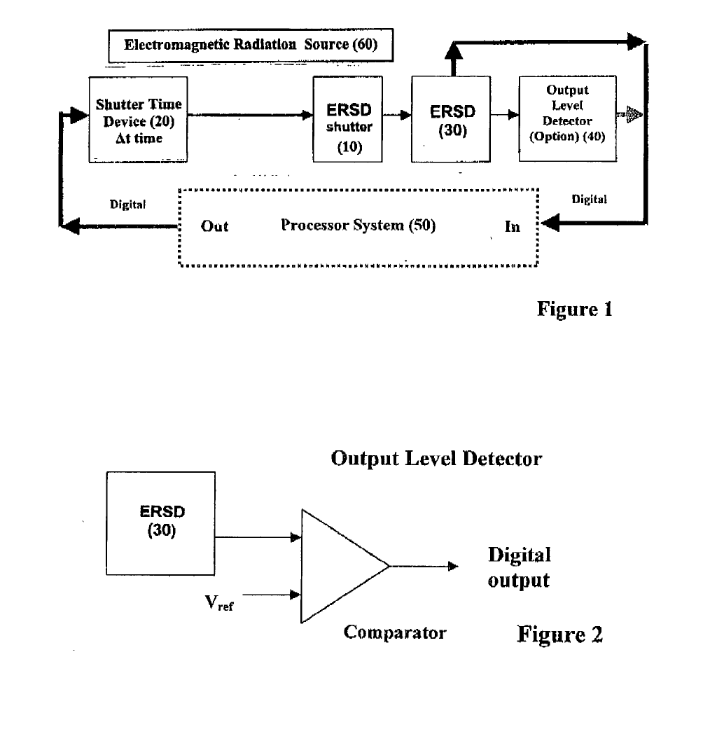

Referring now to FIGS. 1-6, the system according to an embodiment of the present invention comprises:

An electromagnetic radiation source 60 (e.g. LEDs of different colors);

A At shutter speed controller 20;

An electromagnetic radiation sensitive device (ERSD) 30 (e.g. digital or analog camera), and an ERSD shutter 10;

An output level detector 40;

A data processor system 50; and

An illumination region (where the test object is disposed).

The invented method of electromagnetic radiation measurement may be used in the system in accordance with the invention shown in FIG. 1. The system comprises a closed chain of the following functional units:

1. A processor (computer) 50 that controls the shutter speed device 20 (see thick arrow in FIG. 1).

2. The output of the shutter speed controls the Δt shutter speed device 10.

3. The electromagnetic radiation source illuminates a test object disposed in an illumination region 60.

4. An Electromagnetic Radiation Sensitive Device (ERSD) 30 receives modified (e.g. reflected, transmitted, diffused, etc.) electromagnetic radiation from the test object.

5. The ERSD output is digitized if the output is an analog signal, and

6. The digitized ERSD output is read by the processor system 50 (see thick arrow in FIG. 1).

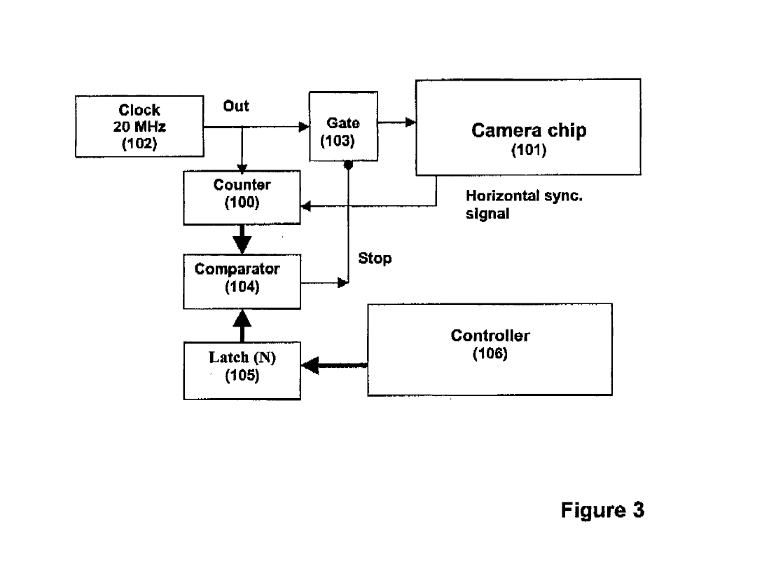

Referring now to FIG. 3 the shutter speed controller comprises the following functional blocks and operations:

1. The horizontal sync. pulse resets the Counter 100.

2. When the Counter 100 is reset, the camera 101 input clocks 102 is halted (by the Stop signal) in the Gate 103.

3. The Counter 100 starts counting clock 102 pulses.

4. The Comparator 104 detects the event of the Counter 100 reaching the count value N, placed in the Latch 105 by the Controller 106.

5. The Stop signal is removed and clocking of the camera 101 continues until the next hor. sync. pulse.

6. The loop 1-5 is then repeated.

By this system, the shutter speed can be adjusted to obtain a constant Target value from the ERSD. The setting of a Δt shutter speed will vary for varying test objects and is used as a measure for the electromagnetic radiation received from the test object by the ERSD.

Spectral information of the electromagnetic radiation from the test object can be obtained by either using electromagnetic radiation sources with different spectral emission or filtering a broadband electromagnetic radiation source before the electromagnetic radiation reaches the (broad-band) ERSD. LED colors can include the visual spectrum, as well as the Near Oared and the Near Ultra Violet spectral range.

The specific units of an embodiment of the system according to the invention will now be described in further detail:

1. The processor 50 is able to control the shutter speed device 20 by the following method when the camera chip in use issues a horizontal synchronization signal whenever the camera has finished an exposure of a line of pixels (shutter speed).

a) The horizontal synchronization signal resets a counter register to zero.

b) When the counter register is reset the camera input clock is halted (by a Stop signal).

c) The counter register is incremented with the rate of the camera clock pulses.

d) A comparison detects tie event of the counter register reaching the count value N, said value N is placed in a register by the processor.

e) The Stop signal is removed when this occurs and clocking of the camera chip continues until the next horizontal synchronization signal.

f) The loop a) to e) is then repeated.

The aforementioned steps of the method for adjusting the shutter speed can preferably be implemented in an ASIC (Application Specific Integrated Circuit) circuit, programmable logic arrays and similar devices, etc., which has an internal set of functional blocks and interconnections as shown in FIG. 3.

2. The electromagnetic radiation source 60 may be any one of

a) Electromagnetic radiation emitting diodes;

b) Incandescent lamps;

c) Gas discharge lamps;

d) Lasers;

e) Masers;

f) X-ray sources; or

g) γ-ray sources, etc.

The electromagnetic radiation from the electromagnetic radiation source can be spectrally filtered if necessary.

3. A test object generally disposed in an illumination region receives electromagnetic radiation from the electromagnetic radiation source 10. Modified (e.g. reflected, transmitted re-transmitted or diffused) electromagnetic radiation from the test object is received by the Electromagnetic Radiation Sensitive Device (ERSD) 30.

4. The ERSD 30 generally comprises an electromagnetic radiation detector and necessary support circuits and optics. Possible electromagnetic radiation detectors comprise:

a) A CCD camera chip

b) A CMOS camera chip

c) All pixel line by pixel line exposable electromagnetic sensitive devices

5. The processor system 50 is able to read the output from the ERSD 30. If the output is an analog signal (voltage or current), this is transformed into a digital signal. This can be done in one of several ways:

a) A comparator can be used, as illustrated in FIG. 2.

b) The voltage or current can be converted into pulses where the pulse rate increases (or decreases) when the voltage or current increases. This can be done by using a voltage (or current)-to-frequency converter. The processor can then measure the time between the pulses (by using its internal clock) and thus digitize the ERSD output signal.

c) An Analog-to-Digital Converter (ADC) can be used.

6. The processor system 50 receives the output signal from the ERSD 30.

a) If the digitizing method illustrated in FIG. 2 is applied, the following procedure may be used:

V_{ref} is adjusted to a suitable output Target value inside the ERSD output range.

The processor 50 adjusts the output of the shutter speed controller 20 according to the Successive Approximation Method (SAM) described below.

b) If a camera 30 with digital output is applied, the following procedure may be used:

A digital Target output value T is selected at a suitable value inside the ERSD output range.

The processor 50 adjusts a Δt shutter speed according to the Successive Approximation Method (SAM) described below.

The fastest way of searching for the electromagnetic radiation level of an unspecified test object is by using the binary Successive Approximation Method (SAM). We will use the SAM when:

a) The relationship between input and output is unknown, or

b) The relationship between input and output is linear, or

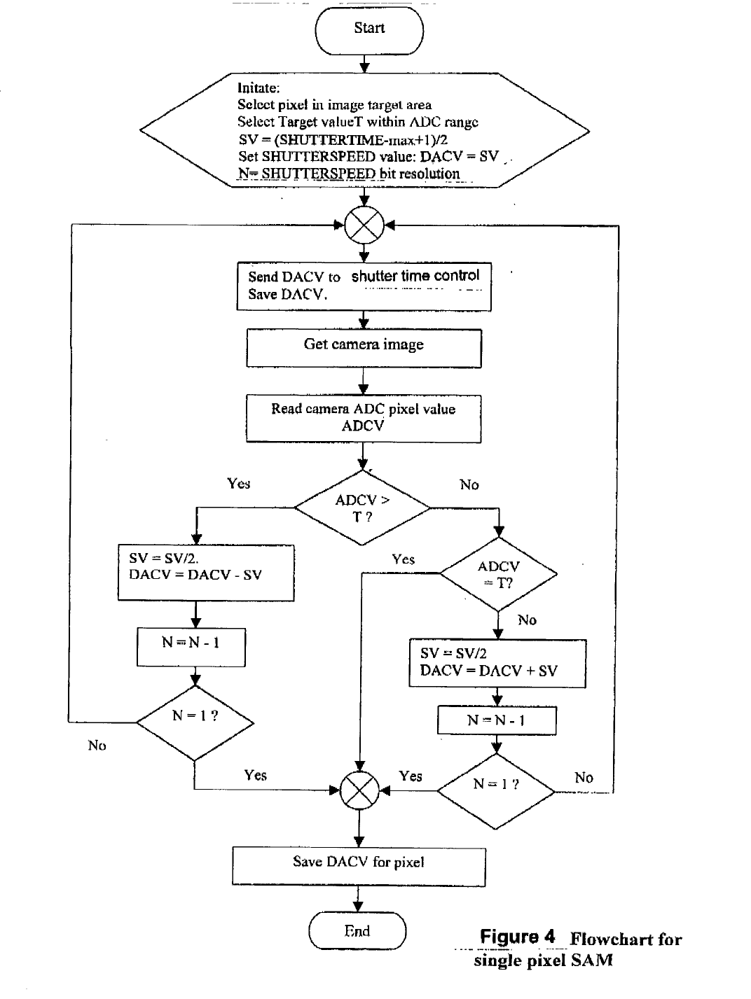

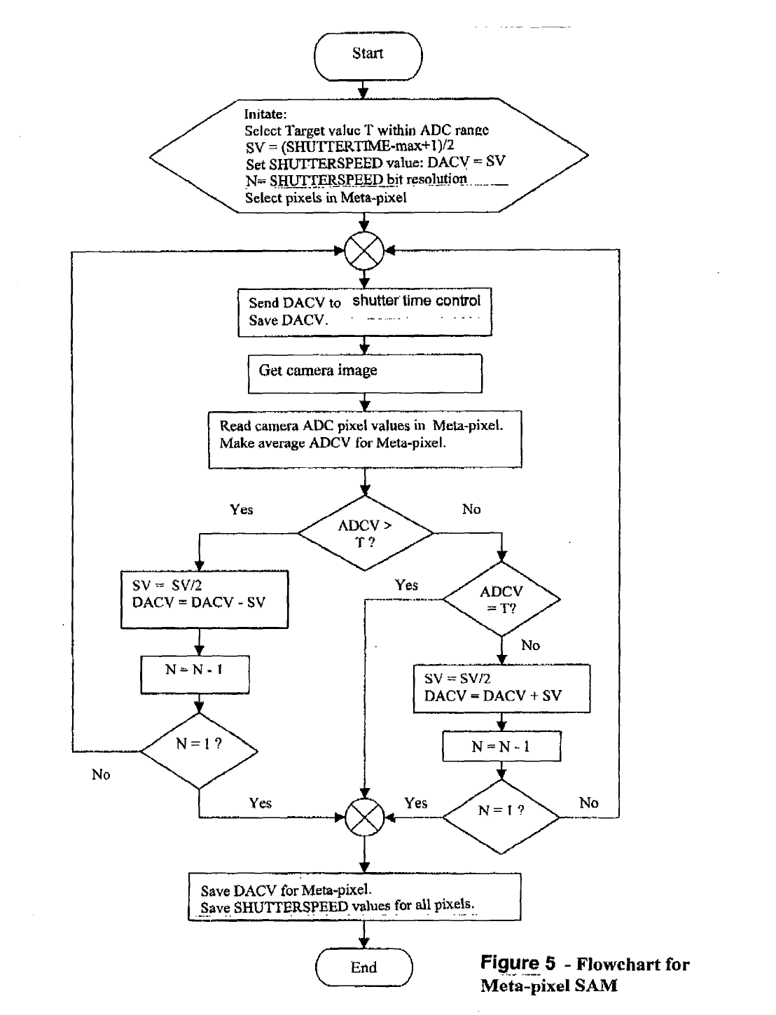

c) The relationship between input and output is non-linear but monotonous increasing or decreasing. The SAM procedure may be described as follows (cf. flowcharts in FIGS. 4 and 5):

1. An output Target value T of the ERSD is defined. If a digital camera system is used T can be any output value of the output range for the system, but preferably a value in the middle of its range. A single pixel output, or the average of a set of pixel outputs can be used as Target value. See details below. If an ERSD with analogue output, connected as shown in FIG. 2, is used the V_{ref} is adjusted to a suitable value (preferably in the middle of the ERSD response range).

2. An initial Step Value (SV=Δt) of the shutter speed is defined as the maximum value +1 of the shutter speed divided by two. If the shutter speed has 10-bit resolution its maximum value will be 1023 and the initial SV will be 512.

3. The initial output of the shutter speed is set equal to SV and a Δt time shutter speed value corresponding to SV is transferred to the shutter speed control device.

4. The steps below will be repeated N−1 times. N is the number of binary digits of the shutter speed. (If the shutter speed has 10 bit resolution N will be equal to 10).

The following loop is executed:

5. The current Δt time shutter speed corresponding to the input shutter speed value is transferred to the shutter speed controller 20 and the current shutter speed value output is used as the V_{ref} and the resulting output from the ADC is measured by comparing.

6. If the ADC value is higher than T then:

The SV is divided by 2

The new SV value is subtracted from the current shutter speed output value and corresponding Δt time shutter speed is transferred.

The loop continues (N−1 times)

If the ADC value is lower than T then:

The SV is divided by 2

The new SV value is added to the current shutter speed output value and the corresponding Δt time shutter speed is transferred.

The loop continues (N−1 times)

If the ADC value is equal to T then (not used if the ADC has one bit output range):

The loop is terminated.

Loop end here

7. After the loop is terminated the current (final) setting of the shutter speed is recorded and used as a measure of the electromagnetic radiation-value.

Each time the steps 5 and 6 are repeated the accuracy is improved by one binary digit (bit). To obtain an accuracy of 1/1024 in the saved illuminance value a maximum of ten illuminance adjustments and image recordings have to be made. Most digital camera circuits can record around 10 images per second or more, thus enabling us to obtain an accurate electromagnetic radiation measurement in about one second or less.

The best mode embodiment of the invention comprises the system depicted in FIG. 1 where the shutter time adjustments are performed with an electronic device implementation of the steps and functional blocks depicted in FIG. 2.

Target Output Value Based on more than One Pixel

More than one pixel can be used to define a target output value from the camera. By letting the summed or averaged output value from a group of pixels represent a “meta-pixel” the same Target search procedure can be applied upon this “Meta-pixel” as on a single pixel. If the test object is a relatively homogenous surface, like a smooth white or colored area, the pixel values of the ADC camera output from this area will only vary within a limited range. If the pixel value range is narrow i.e. within a near-linear part of the response function the images recorded from the search-procedure described above can be used to adjust each pixel value to compute the shutter speed-value that yields the Target value. This can be done by linear approximation.

If the pixel value range is larger, they should be divided in subgroups, each lying within a near-linear part of the response function. The average of the main sub-group should be used to define the Target value in the search-procedure described above. For increased accuracy extra images with target values for each group can be recorded.

(Note: Even if the surface of the test object is absolute homogenous the pixel outputs from the test object image will vary, due to unavoidable irregularities in camera pixel sizes, homogeneity of illumination, camera optics, etc.)

Since the “meta-pixel” is an average of many pixels its numeric resolution better than that of the ADC output for a single pixel. Or opposite: If the ADC output is 10 bits of higher we can only save the 8 most significant bits and will still obtain high accuracy for the “meta-pixel” value.

Calibration

The relationship between the ADC outputs of the camera and the shutter speed settings of shutter speed can be obtained as follows: A Reference Test Object is used, preferably a white surface if reflectance is measured, or a clear object if transmittance or electromagnetic radiation scattering is measured. For each ADC value the corresponding shutter speed value is recorded in a calibration-table. (If the transfer function is a smooth curve only a limited number of measurements have to be made to establish the calibration table).

Depending on the setting of camera control parameters the relationship may be similar to the function for electromagnetic radiation from a white object presented in FIG. 3. If the relationship between shutter speed-value and electromagnetic radiation intensity is close to linear (or linear) this calibration curve can be later used to compute the reflectance for all test object (inside the measurement-range).

Any suitable ERSD device used in the present invention will, in addition to the target value output, include a background signal due to environmental conditions such as temperature and physical effects in the device it self as for example dark-currents etc. The measured target values have to be compensated for this background effect to maintain the high resolution of the measurements. This can be done by for example recording an image without the target object in the system thereby subtracting said recorded image from the images of the target object. The same effect can be achieved by taking a succession of images and then determine the background signal from this series of images.

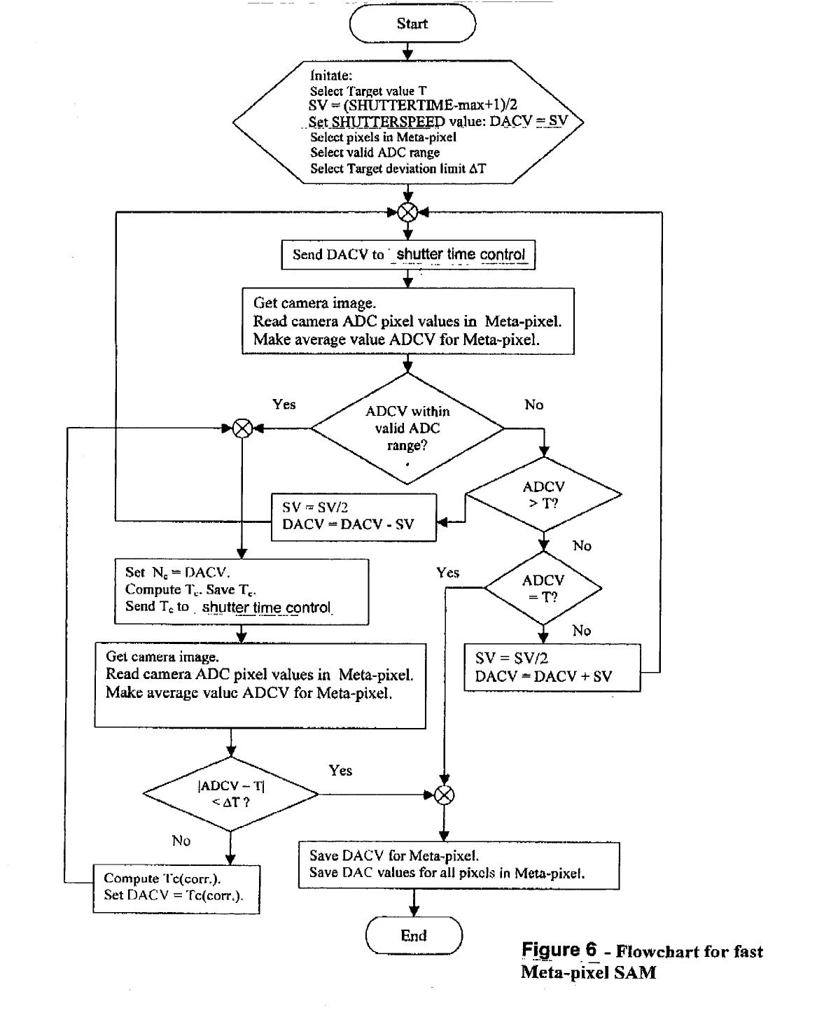

Speeding up the Successive Appmoximation Method (cf. FIG. 6) (Note: This Method Cannot be used for a Single-Bit ADC True. Like the One Shown in FIG. 2).

When the relationship between shutter speed input and ADC output is calibrated for an illuminated reference object (usually a white object) then the calibration table can be used to obtain a result quickly by the processor system. Reading from tables in the processor memory is normally much faster than adjusting the shutter speed and subsequently recording the output from the ERSD.

What is claimed is:

1. A system for digitizing electromagnetic radiation measurements by controlling adjustments of a shutter speed of an electromagnetic radiation sensitive device, said system having an electromagnetic source illuminating an illumination region, said system obtaining a constant or near constant signal from said electromagnetic radiation sensitive device, the system comprising:
An electromagnetic radiation source configured to illuminate an illumination region having a test object by a plurality of electromagnetic radiation signals;
An electromagnetic radiation sensitive device configured to record the plurality of electromagnetic radiation signals generally modified by the test object in the illumination region and transmit an output signal corresponding to the modified plurality of electromagnetic radiation signals;
A data processor system configured to receive the output signal and generate a controlling signal; and
A shutter speed controller, receivably connected to the data processor system via the controlling signal, the shutter speed controller controlling the adjustments of the shutter speed (exposure time of the camera), whereby the recorded electromagnetic radiation signals are adjustably controllable such that said output signal is constant.

2. The system in accordance with claim 1, wherein the electromagnetic radiation sensitive device is a digital cameras.

3. The system in accordance with claim 1, wherein the electromagnetic radiation sensitive device is a digital video camera.

4. The system in accordance with claim 1, wherein the electromagnetic radiation sensitive device is an analog camera comprising a controllable shutter device.

5. The system in accordance with claim 4, additionally comprising an output level detector receivably connected to the electromagnetic radiation sensitive device and configured to provide a digital signal representative of the output signal of the electromagnetic radiation sensitive device.

6. The system in accordance with claim 1, wherein said modified electromagnetic radiation signals are modified by reflection from, and/or transmission through, and/or diffusion by, a said test object in said illumination region.

7. The system in accordance with claim 1, wherein said output signal is an analog signal and wherein said analog output signal is transmitted to an output level detector; said output level detector by means of an adjustable reference voltage Vref yielding a 1 bit digital output signal.

8. The system in accordance with claim 1, wherein said output signal is a digital signal.

9. The system in accordance with claim 1, wherein said electromagnetic radiation detector comprises a CCD camera chip or equivalent.

10. The system in accordance with claim 1, wherein said electromagnetic radiation detector comprises a CMOS camera chip or equivalent.

11. The system in accordance with claim 1, said processor system reading the output from the Electromagnetic radiation Sensitive Device.

12. The system in accordance with claim 1, wherein the Electromagnetic radiation Sensitive Device (ERSD) output is an analog signal (voltage or current).

13. The system in accordance with claim 12, wherein said analog signal is transformed into a digital signal by means of a comparator.

14. The system in accordance with claim 12, wherein said analog signal is converted into pulses where the pulse rate increases or decreases when the voltage or current increases; said pulse rate increase or decrease being accomplished by using a voltage (or current)-to-frequency converter; said processor subsequently measuring the time between pulses (e.g. by using its internal clock) and thus digitizing the ERSD output signal.

15. The system in accordance with claim 12, wherein said analog signal is transformed into a digital signal by means of an Analog-to-Digital Converter (ADC).

16. A system for digitizing electromagnetic radiation measurements by controlling adjustments of a shutter speed of an electromagnetic radiation sensitive device, the emission of an electromagnetic radiation source illuminating at least one test object in an illumination region, in order to obtain a constant or near-constant signal from an electromagnetic radiation sensitive device, the system comprising:
A data processor system configured to generate a controlling signal;
A shutter speed controller responsive to the controlling signal;
A shutter responsive to the shutter speed controller;
An illumination region, including a test object, illuminated by the electromagnetic radiation source; and
An electromagnetic radiation sensitive device configured to image the electromagnetic radiation modified by the test object and communicate an output signal representative of the modified electromagnetic radiation to the data processor system, whereby the modified electromagnetic radiation signal is adjustably controllable such that the output signal is constant.

17. The system in accordance with claim 16, wherein the electromagnetic radiation sensitive device images a plurality of electromagnetic radiation signals generally modified by the test object.

18. The system in accordance with claim 16, wherein said output signal is an analog signal and wherein said analog output signal is transmitted to an output level detector; said output level detector by means of an adjustable reference voltage Vref yielding a 1 bit digital output signal.

19. The system in accordance with claim 16, wherein said output signal is a digital signal.

20. The system in accordance with claim 16, wherein said electromagnetic radiation detector comprises a CCD camera chip or equivalent.

21. The system in accordance with claim 16, wherein said electromagnetic radiation detector comprises a CMOS camera chip or equivalent.

22. The system in accordance with claim 16, wherein the Electromagnetic radiation Sensitive Device (ERSD) output is an analog signal (voltage or current).

23. The system in accordance with claim 21, wherein said analog signal is transformed into a digital signal by means of a comparator.

24. The system in accordance with claim 21, wherein said analog signal is converted into pulses where the pulse rate increases or decreases when the voltage or current increases; said pulse rate increase or decrease being accomplished by using a voltage (or current)-to-frequency converter; said processor subsequently measuring the time between pulses (e.g. by using its internal clock) and thus digitizing the ERSD output signal.

25. The system in accordance with claim 21, wherein said analog signal is transformed into a digital signal by means of an Analog-to-Digital Converter (ADC).

26. A method of digitizing electromagnetic radiation levels by successive approximation to measure an electromagnetic radiation value, the method comprising:
Identifying an output target value of an electromagnetic radiation sensitive device receiving electromagnetic radiation signals modified by a test object;
Defining an initial step value of an analog to digital converter (ADC) connected to the electromagnetic radiation sensitive device;
Setting the initial step value to be the value of the input of a shutter speed device which corresponds to a Δt time controlling a shutter speed, wherein the shutter speed has an N bit resolution;
Repeating one or more shutter speed adjustments and corresponding Δt time shutter speeds based on a relationship between the shutter speed output value and Mile ADC value of the output target value for up to N−1 iterations while calculating corresponding Δt time adjustments until the ADC value is equal to the output target value when the adjustments are completed; and
Identifying the final shutter speed output value (final corresponding Δt time adjustment value) as a measure of the value of the electromagnetic radiation signals.

27. The method in accordance with claim 26 wherein the adjustments of the shutter speed value include:
If the ADC value is greater than the output target value, dividing the step value by two and subtracting the new step value from the current shutter speed output value and use a corresponding Δt time shatter speed, and
If the ADC value is less than the output target value, dividing the step value by two and adding the new step value to the current shutter speed output value and use a corresponding Δt time shutter speed.

28. The method in accordance with claim 26, wherein the output target value is selected to be in the middle of a response range of the electromagnetic radiation sensitive device.

29. A method of digitizing electromagnetic radiation measurements by controlling adjustments of a shutter speed of an electromagnetic radiation sensitive device, the emission of an electromagnetic radiation source illuminating an illumination region containing a test object, to obtain a constant or near constant signal from the electromagnetic radiation sensitive device, the method comprising:
Illuminating an illumination region by a plurality of electromagnetic radiation signals;
Modifying the plurality of electromagnetic radiation signals;
Recording the plurality of modified electromagnetic radiation signals;
a Transmitting an output signal corresponding to the plurality of modified electromagnetic radiation signals; and
Controlling the operation of a shutter based on the output signal, whereby the received electromagnetic radiation signals ate adjustably controllable such that the output signal is constant.

30. The method in accordance with claim 29, wherein the output signal is a digital signal.

31. The method in accordance with claim 29, wherein the output signal is an analog signal and the method additionally comprises converting the analog signal to a digital signal.

32. The method in accordance with claim 29, additionally comprising generating a controlling signal based on the output signal so as to control the operation of the shutter speed.

33. The method in accordance with claim 29, wherein said processor system receives said output signal from the ERSD and a digitizing method comprises:
Adjusting Vref to a suitable output Target value inside the ERSD output range; and
By means of said processor adjusting the shutter speed time output according to a Successive Approximation Method (SAM).

34. An Electronic Device for Controlling a shutter speed adjustment comprised in an electronic radiation sensitive device (101), comprising:
a Clock (102);
a Gate (103);
a Counter (100);
a Comparator (104);
a Latch (105);
a Controller (106);
Whereby said Counter (100) receivably is connected to a control signal from said electromagnetic radiation sensitive device (101) that is resetting said Counter (100) that starts counting said clock (102) pulses; a Stop signal is receivably connected to said electromagnetic radiation sensitive device (101) via said Gate (103) that halts said clock (102) pulses to clock said electromagnetic radiation sensitive device (101) whenever said Stop signal is received; said Gate (103) is receivably connected to said Comparator (104) that issue said Stop signal whenever a content of said Counter (100) is equal to a content of said Latch (105) receivably connected to said Controller (106).

35. An Electronic Device according to claim 34, wherein the Electronic Device is an ASIC circuit.

36. An Electronic Device according to claim 34, wherein the Electronic Device is a FPGA circuit.

37. An Electronic Device according to claim 34, wherein the Electronic Device is a FPLD circuit.

38. An Electronic Device according to claim 34, wherein the Electronic Device is a PLD circuit.

39. An Electronic Device according to claim 34, wherein tie Electronic Device is a CPLD circuit.

40. Au Electronic Device according to claim 34, wherein the Electronic Device is a fall custom VLSI circuit.
